(19) United States
(12) Patent Application Publication
(10) Pub. No.: US 2009/0099487 A1
(43) Pub. Date: Apr. 16, 2009

(54) High-intensity focused ultrasound probe movement control device

(76) Inventors: Rodrigo Chaluisan, Charlotte, NC (US); George M. Suarez, Miami, FL (US)

Correspondence Address: REED SMITH LLP, 2500 ONE LIBERTY PLACE, 1650 MARKET STREET, PHILADELPHIA, PA, 19103, US

(21) Appl. No.: 11/974,284

(22) Filed: Oct. 12, 2007

(51) Int. Cl. A61N 7/02

(52) U.S. Cl. 601/3

(57) ABSTRACT

The present invention pertains to a high-intensity focused ultrasound probe movement control device.

### TECHNICAL FIELD OF THE INVENTION

The invention generally relates to a high-intensity focused ultrasound probe movement control device capable of making small adjustments that in turn adjust a transrectal ablation probe.

### BACKGROUND OF THE INVENTION

Prostate cancer is one of the most common cancers diagnosed in men. People diagnosed with prostate cancer have several options for treating and/or managing the cancer. These options include watching and waiting, surgery, radiation, and thermal or hormonal therapies. Gardner et al., Clinical Genitourinary Cancer, Vol. 4, No. 3, 187-192 (2005). One thermal therapy of interest is high-intensity focused ultrasound (HIFU). HIFU is a non-invasive therapy option that results in the ablation of diseased tissue using ultrasound waves. The transducer of a transrectal ablation probe concentrates ultrasound energy at a focal point generating temperatures that can exceed 100° C. resulting in complete coagulative necrosis and the destruction of tissue. The transrectal probe has been used for such diseases as cancer and also for benign prostatic hyperplasia.

The prostate gland is a small structure surrounded by other organs and tissue. The goal of treatment of a tumor of the prostate with a transrectal ablation probe is the destruction of only the diseased tissue, which requires steady control of the probe. A standard probe can weigh between 10 and 40 pounds and be up to 36 inches long. For example, the Sonablate® 500 probe, from Focus Surgery, Inc., weighs approximately 20 pounds. The probe is about 22 inches long. The probe can also have varying focal length transducers, wherein the focal length can be anywhere from approximately 2 cm to approximately 5 cm, such that precise movement of the probe is critical for creating the precise dimension necessary for abating the diseased tissue. A physician can have a difficult time accurately maintaining the location of the probe due to its cumbersome weight. Therefore, a device that can hold a transrectal ablation probe and allow for precise movement is needed. Having a device that can make precise adjustments will help with accuracy of the ablation process and ultimate successful outcome, and minimize negative outcomes, such as ablating healthy tissue.

### SUMMARY OF THE INVENTION

The present invention pertains to a HIFU probe movement control device having a first controlled directional member capable of moving along a first axis and a second controlled directional member capable of moving along a second axis. The first controlled directional member is coupled with the second controlled directional member. The HIFU probe movement control device may also have an attachment device that is configured and dimensioned to couple the HIFU probe movement control device with a HIFU probe. Further, the HIFU probe movement control device may have a third controlled directional member capable of moving along a third axis or an articulate arm having an adjustable length along a third axis.

### BRIEF DESCRIPTION OF THE DRAWINGS

FIG. 1 shows an embodiment of the present invention;

FIG. 2 shows an alternative view of an embodiment of the present invention; and

FIG. 3 shows an embodiment of the invention wherein a transrectal ablation device is coupled to the device.

### DETAILED DESCRIPTION

Reference will now be made in detail to embodiments of the invention. While the invention will be described in conjunction with these embodiments, it will be understood that they are not intended to limit the invention to those embodiments. On the contrary, the invention is intended to cover alternatives, modifications, and equivalents, which may be included within the spirit and scope of the invention as defined by the appended claims.

The present invention pertains to a HIFU probe movement control device 10 for use with a transrectal ablation device to enable accurate, controlled movements necessary for destruction of diseased tissue only for the successful treatment of prostate diseases. The term “diseased” includes, but is not limited to benign tumors, malignancies, hypertrophy, and irregular or abnormal tissue or cells.

The HIFU probe movement control device 10 has a first controlled directional member 120 capable of moving along a first axis and a second controlled directional member 130 capable of moving along a second axis. The HIFU probe movement control device 10 also has an attachment device 40 that is configured and dimensioned to couple with the HIFU probe movement control device 10 and a probe 160.

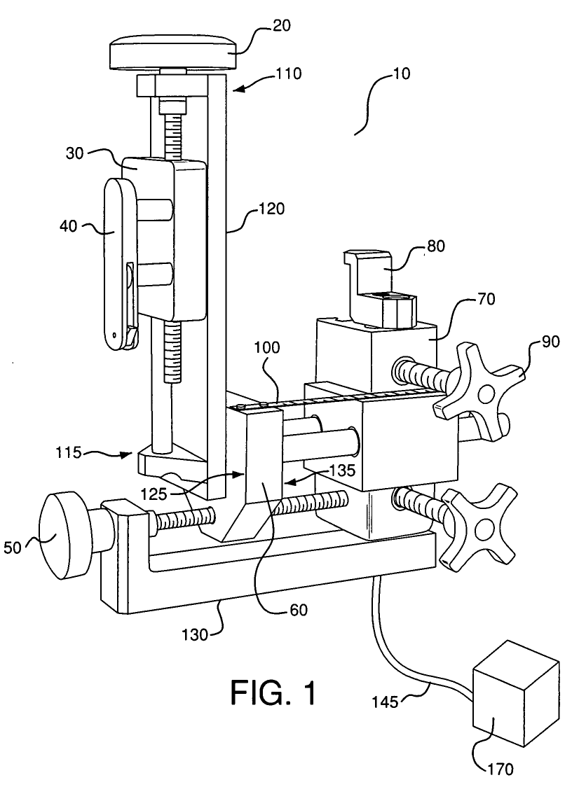

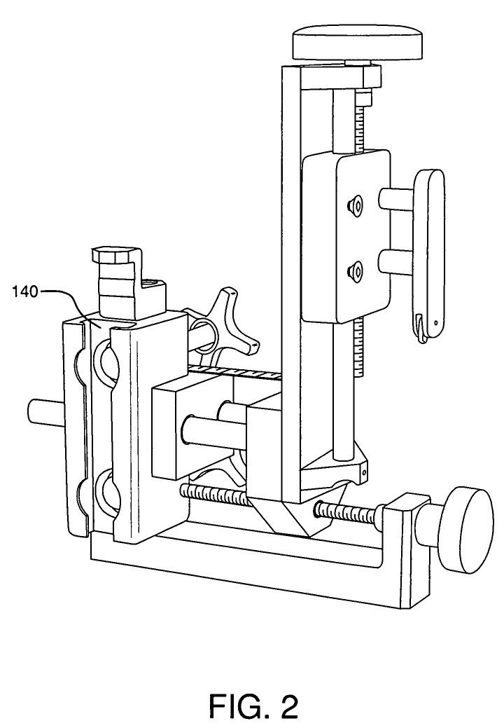

According to an embodiment as shown in FIGS. 1 and 2, the HIFU probe movement control device 10 has a first controlled directional member 120 having a first movement generator 20 and a first moveable member 30. The first controlled directional member 120 has a first end 110 and a second end 115. Rotation, movement, activation, or adjustment (herein collectively referred to as “rotation” or “rotating”) of the first movement generator 20 results in the movement of the moveable member 30.

The first moveable member 30, in this instance a vertical adjustment plate is configured and dimensioned to couple with the first movement generator 20 such that rotation of the first movement generator 20 causes the first moveable member 30 to move along a first axis with respect to the first movement generator 20. The first moveable member 30 may also be integral with the first movement generator 20 or a part of the first movement generator 20.

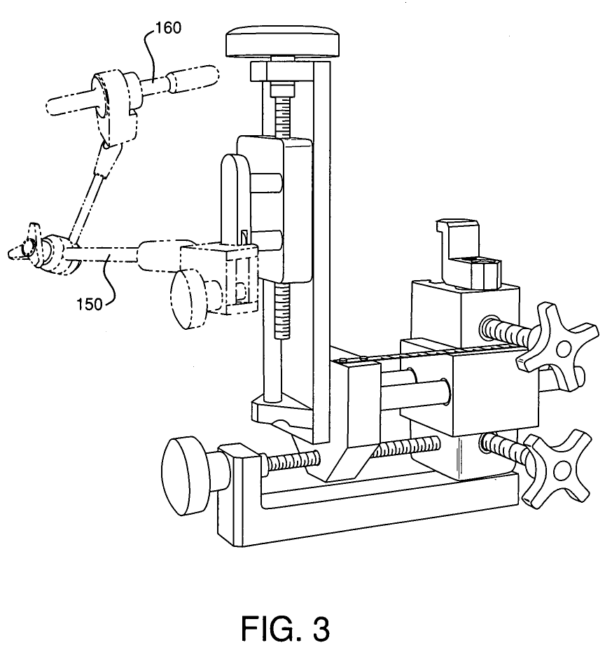

An attachment device 40 is coupled with the first moveable member 30, wherein the device attachment 40 is capable of being coupled with an articulated arm 150 or a probe 160. The probe 160 in FIG. 3 is a representation of a probe; the size and shape of which may vary depending upon many factors including, but not limited to the designer and/or manufacturer of the probe. The probe 160 may be a dilator or a HIFU probe, preferably a HIFU probe. The articulated arm 150 may be coupled with a probe 160 (See FIG. 3). The device attachment 40 may removably or fixedly attach to an articulated arm 150 or a probe 160. Removably attached includes, but is not limited to a clamp, a screw, a vice, a latch, or mechanism known in the art for removably attaching two apparatuses together. The term “fixedly attached” includes, but is not limited to an adhesive, glue, bonded, integral, or any mechanism known in the art for securing two apparatuses together. The attachment device 40 may have a latch, or screw, or any other mechanism known in the art for coupling the articulated arm 150 or the probe 160 with the attachment device 40.

The articulated arm 150 may also have an adjustable length such that the articulated arm 150 can be shortened or lengthened along an axis. The adjustable length of the articulated arm 150 may be generated by a controlled directional member, or by any other mechanism known in the art.

The HIFU probe movement control device 10 also has a second controlled directional member 130 having a second movement generator 50 and a second moveable member 60. The second moveable member 60 has a first side 125 and a second side 135. Rotation of the second movement generator 50 results in the movement of the second moveable member 60. The second moveable member 60 may also be integral with the second movement generator 50 or a part of the second movement generator 50.

The first controlled directional member 120 is configured and dimensioned to couple with the second moveable member 60 such that the first controlled directional member 120 is capable of being moved along a second axis with respect to the second moveable member 60. The second moveable member 60 may be coupled toward or near the second end 115 of the first controlled directional member 120

The HIFU probe movement control device 10 may also have a third controlled directional member having a third movement generator that can move a probe along a third axis. The third controlled directional member may be coupled with the first controlled directional member 120 or the second controlled directional member 130. The third controlled directional member may also be coupled with the first moveable member 30 or the second moveable member 60.

The HIFU probe movement control device 10 may have a measurement device 100. The measurement device 100 may be an infrared, digital, or laser measurement tool, a caliper, a ruler, or any other measurement tool known in the art. A measurement device 100 may be coupled with the first controlled directional member, the second controlled directional member, and/or the third controlled directional member. The measurement device 100 is capable of determining the amount of movement of the first controlled directional member, the second controlled directional member, and the third controlled directional member, which in turn can measure the movement of the probe 160.

The HIFU probe movement control device 10 may be coupled with a control unit 170 via a wire 145 or other means known in the art for delivering a signal to/from the HIFU probe movement control device 10, including but not limited wireless communication such as radio frequency, microwave, and infrared. The control unit 170 is capable of rotating or activating the first movement generator, the second movement generator, and/or the third movement generator, which in turn moves the first moveable member, the second moveable member, and/or the third moveable member. The control unit 170 may rotate or activate the first movement generator, the second movement generator, and the third movement generator at the same time or at different times with respect to each other.

The first movement generator 20, the second movement generator 50, and the third movement generator may be, for example, a screw, a rack and pinion, a shaft and motor, a ratchet, a piston, hydraulics, and any other mechanism known in the art that may be used to generate movement of the first moveable member 30 and the second moveable member 60.

The HIFU probe movement control device 10 may also have a securing mechanism 70 that couples the HIFU probe movement control device 10 with a platform that may include, but is not limited to a table, stand, gurney, bed, examination table, the floor, and operating table. The HIFU probe movement control device 10 may removably or fixedly attach to a platform by any of the mechanisms described herein or by any mechanism known in the art. The securing mechanism 70 may be a bracket, a clamp, a vice, or any other device that secures the HIFU probe movement control device with a platform. The securing mechanism 70 may have a stop 80, at least one cross screw 90, and a guide 140. The cross screw 90 allows the HIFU probe movement control device 10 to be secured to any platform by rotating the cross screw 90 causing it to clamp to the platform with the HIFU probe movement control device 10. The guide 140 may receive any portion of a platform to secure the HIFU probe movement control device 10 to the platform. Once the a portion of a platform is inserted into the guide 140 the cross screw 90 may be rotated to secure the HIFU probe movement control device 10 to the platform. The stop 80 prevents the HIFU probe movement control device 10 from sliding or otherwise moving with respect to any platform it may be coupled with. The stop 80 may be of any size necessary for preventing the HIFU probe movement control device 10 from moving with respect to a platform is may be couple with; preferably the stop 80 is about 1 to about 6 inches in height.

Although the present invention has been described in terms of specific embodiments, changes and modifications can be made out without departing from the scope of the invention which is intended to be defined only by the scope of the claims.

What is claimed is:

1. A high-intensity focused ultrasound probe movement control device, comprising:
a first controlled directional member capable of moving along a first axis in a controlled fashion;
a second controlled directional member capable of moving along a second axis in a controlled fashion; and
an attachment device configured and dimensioned to couple the movement control device with a high-intensity focused ultrasound probe,
wherein action of each of the controlled directional members causes motion of the high-intensity focused ultrasound probe in the direction of each of the controlled directional members' respective axes.

2. The device of claim 1, wherein the first controlled directional member comprises a first movement generator and a first moveable member, and wherein the second controlled directional member comprises a second movement generator and a second moveable member.

3. The device of claim 2, wherein the first movement generator and the second movement generator are selected from the group consisting of a screw, a rack and pinion, a shaft and motor, a ratchet, a piston, and hydraulics.

4. The device of claim 1, further comprising:
an articulated arm,
wherein the articulated arm is configured and dimensioned to couple with the attachment device, and
wherein the high-intensity focused ultrasound probe is configured and dimensioned to couple with the articulated arm.

5. The device of claim 1, further comprising:
a platform, and
a securing mechanism, wherein the securing mechanism is configured and dimensioned to couple with the platform,
wherein the securing mechanism comprises at least one cross screw and a stop,
wherein the cross screw is capable of securing the device to the platform, and
wherein the stop is capable of preventing the device from sliding along the platform.

6. The device of claim 5, wherein the platform is selected from the group consisting of an examination table, an operating table, and a bed.

7. The device of claim 1, 4, or 5, further comprising:
at least one measurement device,
wherein the measurement device is capable of measuring the movement of at least one of the first controlled directional member or the second controlled directional member.

8. The device of claim 1, wherein the probe is a high-intensity focused ultrasound probe for ablating diseased tissue of a prostate gland.

9. The device of claim 1, further comprising:
a third controlled directional member capable of moving along a third axis in a controlled fashion,
wherein the third controlled directional member is coupled with the first controlled directional member or the second controlled directional member.

10. The device of claim 9, wherein the third controlled directional member comprises a third movement generator and a third moveable member.

11. The device of claim 9 or 10, further comprising:
at least one measurement device,
wherein the measurement device is capable of measuring the movement of third controlled directional member.

12. The device of claim 10, wherein the third movement generator is selected from the group consisting of a screw, a rack and pinion, a shaft and motor, a ratchet, a piston, and hydraulics.

13. A method of treating a patient having a prostate disease, comprising:
positioning a high-intensity focused ultrasound probe substantially adjacent to a diseased tissue,
adjusting the high-intensity focused ultrasound probe using a movement control device, wherein the movement control device comprises,
a first controlled directional member capable of moving along a first axis in a controlled fashion,
a second controlled directional member capable of moving along a second axis in a controlled fashion, and
a attachment device configured and dimensioned to couple the movement control device with the high-intensity focused ultrasound probe,
wherein action of each of the controlled directional members causes motion of the high-intensity focused ultrasound probe in the direction of each of the controlled directional members' respective axes; and
ablating the diseased tissue with the probe.

14. The method of claim 13, wherein the first controlled movement generator comprises a first movement generator and a first moveable member, and wherein the second controlled movement generator comprises a second movement generator and a second moveable member.

15. The device of claim 13, wherein the first movement generator and the second movement generator are selected from the group consisting of a screw, a rack and pinion, a shaft and motor, a ratchet, a piston, and hydraulics.

16. The method of claim 13, wherein the movement control device further comprises:
an articulated arm,
wherein the articulated arm is configured and dimensioned to couple with the device attachment, and
wherein the high-intensity focused ultrasound probe is configured and dimensioned to couple with the articulated arm.

17. The method of claim 13, wherein the diseased tissue is of a prostate gland.

18. The method of claim 13, wherein the movement control device further comprises:
a third controlled directional member capable of moving along a third axis in a controlled fashion,
wherein the third controlled directional member is coupled with the first controlled directional member or the second controlled directional member.

19. The method of claim 18, wherein the third controlled directional member comprises a third movement generator and a third moveable member.

20. The method of claim 19, wherein the third movement generator is selected from the group consisting of a screw, a rack and pinion, a shaft and motor, a ratchet, a piston, and hydraulics.

21. The method of claim 13, wherein the movement control device further comprises:
at least one measurement device,
wherein the measurement device is capable of measuring the movement of at least one of the first controlled directional member or the second controlled directional member.

22. The method of claim 18, wherein the movement control device further comprises:
at least one measurement device,
wherein the measurement device is capable of measuring the movement of the third controlled directional member.
